(19) United States
(12) Patent Application Publication
(10) Pub. No.: US 2021/0337775 A1
(43) Pub. Date: Nov. 4, 2021

(54) USE OF AN EGG GRAFTED WITH TUMOR CELLS IN ORDER TO STUDY THE ANTI-CANCER EFFECTIVENESS OF IMMUNE THERAPIES IN THE ABSENCE OF IMMUNE EFFECTOR CELLS OTHER THAN THOSE IN THE GRAFTED EGG

(71) Applicant: INOVOTION, LA TRONCHE (FR)

(72) Inventors: Xavier ROUSSET, SAINT MARTIN D'HERES (FR); Emilien DOSDA, CHIRENS (FR); Jean VIALLET, SAINT MARTIN D'HERES (FR)

(73) Assignee: INOVOTION, LA TRONCHE (FR)

(21) Appl. No.: 17/289,198

(22) PCT Filed: Oct. 29, 2019
(86) PCT No.: PCT/FR2019/052572

§ 371 Date: Apr. 27, 2021

(30) Foreign Application Priority Data
Oct. 29, 2018 (FR) 1860000

(51) Int. Cl. A01K 67/027; C12N 5/09; G01N 33/50; A61K 49/00

(52) U.S. Cl. CPC A01K 67/0271; C12N 5/0693; G01N 33/5011; G01N 33/5014; G01N 33/5088; G01N 2333/465; A01K 2207/12; A01K 2227/30; A01K 2267/0331; C12N 2503/02; A61K 49/0008

(57) ABSTRACT

The present invention relates to the use of an embryonated egg model grafted with tumour cells to investigate anti-cancer effectiveness or to screen immunotherapeutic molecules, in the absence of immune effector cells other than those in the grafted egg.

### FIELD OF THE INVENTION

The present invention relates to the field of immuno-oncology and in particular to personalised medicine in order to prescribe to cancer patients the most promising immunotherapy in terms of effectiveness.

### PRIOR ART

Despite already existing therapeutic solutions in cancer treatment (surgery, irradiation, chemotherapy, and targeted therapies), some malignant tumours remained incurable until the discovery of mechanisms by which the immune system can act on the tumour.

These advances in the field have led to the development of a new therapeutic approach called immunotherapy, which seeks to reactivate or stimulate the immune system to specifically attack tumour cells.

One of the interests of immunotherapy is to develop treatments that are not dependent on a given type of cancer, but on the genetic profile and the presence of specific biomarkers in the tumour (PDL-1 type). These treatments thus take account of the profile of the patient and their tumour, a first step towards personalised medicine.

Various courses of action are being contemplated and yielded promising results since 2010:
use of monoclonal antibodies, whether or not associated with cytotoxic molecules,
use of immune checkpoint inhibitors (checkpoints of pathways specifically activated or inhibited in cancer mechanisms) to activate or inhibit some mechanisms involving the immune system in development, such as the use of a CTLA-4 inhibitor (ipilimumab),
stimulation of the immune system to fight tumour cells more effectively, especially with non-targeted immunotherapies, preventive or curative cancer vaccines,
use of adoptive cell therapy, such as CAR-T Cell (Chimeric Antigen Receptor T lymphocytes), which aims at modifying patients' cells in vitro, which are then re-injected into the patient to fight the tumour, with promising results.

Most immunotherapies require the presence of immune cells to be effective, which limits the possibilities for in vitro testing of such molecules. The stages of development are therefore rapidly shifting to animal models, mainly in mice.

The mouse models that spontaneously develop tumours do not have the genetic complexity that exists in patient tumours, which can inhibit or conversely amplify any treatment effect. Thereby, this makes it difficult to extrapolate results to humans.

The first in vivo models used for xenografts were immunodeficient mice, which facilitate development of the tumour which is not attacked by the host immune system. These mouse models were then ‘humanised’ by making transgenic models through the expression of human genes (knock-in), or by grafting human haematopoietic cells into immunodeficient mice. However, these models have several drawbacks, such as the time required to develop the model, which takes several months before any results can be obtained, or the speed of tumour development, which is faster than in humans, this development not being accompanied with chronic inflammation in the tumour environment as it is in humans.

In view of these difficulties, the associated costs and the time required to carry out investigations in a humanised mouse model, there is a need to develop other simpler, faster and more reliable models in order to develop and validate effectiveness of new immunotherapies.

### SUMMARY OF THE INVENTION

The present invention relates to the use of an embryonated bird egg model grafted, in particular at the level of the chorioallantoic membrane (CAM), with tumour cells to evaluate the anti-cancer activity of one or more immunotherapeutic molecule(s), wherein said model excludes the presence of immune effector cells other than those of the grafted egg.

Preferably, the immunotherapeutic molecule is selected from an adoptive cell therapy such as CAR-T, a vaccine, a bi-specific antibody, an immune checkpoint inhibitor such as an anti-PD1, or anti-PDL1, or anti CTLA-4 antibody.

In particular, and when tumour cells are isolated from a cancer patient sample, testing multiple immunotherapeutic molecules allows the determination of which one will be the most promising in terms of cancer treatment effectiveness in that patient.

Within the scope of the use of this embryonated egg, it is also possible to determine, or even quantify, toxicity of the immunotherapeutic molecule(s) tested, both on tumours that have developed from the grafted tumour cells and on the embryo as a whole.

The present invention also relates to a method for evaluating the anti-cancer activity of one or more immunotherapeutic molecule(s), characterised in that it comprises:
grafting tumour cells at the level of the chorioallantoic membrane (CAM) of an embryonated bird egg previously incubated to a stage of development corresponding to CAM formation and equivalent to at least 8 days of development in the chicken embryo,
administrating the immunotherapeutic molecule(s) into the embryonated egg at least 12 hours after grafting,
investigating the effect of the immunotherapeutic molecule(s) thus administered on the tumourigenesis of the tumours which have developed in the grafted embryo, and that it is implemented in the absence of and without adding effector immune cells other than those of the grafted egg.

The present invention is also directed to a method for screening for immunotherapeutic molecules having anti-cancer activity, comprising the following steps of:
grafting tumour cells at the level of the chorioallantoic membrane (CAM) of an embryonated bird egg previously incubated to a stage of development corresponding to CAM formation, and equivalent to at least 8 days of development in the chicken,
administrating the candidate immunotherapeutic molecule(s) into the embryonated egg at least 12 hours after grafting,
investigating the effect of the immunotherapeutic molecule(s) thus administered on the tumourigenesis of the tumours which have developed in the grafted embryonated egg,

said method being implemented in the absence of and without adding effector immune cells other than those of the grafted egg.

The present invention is finally concerned with a method for monitoring a patient or animal with cancer, comprising:
preparing a first embryonated bird egg as described above with tumour cells from said patient or animal at a time T1, and investigating the tumourigenesis of the tumours that develop in this first embryonated egg,
preparing a second embryonated bird egg as described above with tumour cells from the same patient or animal at a time T2, and investigating the tumourigenesis of the tumours that develop in this second embryonated egg,
comparing the tumourigenesis of the tumours that have developed in the first and in the second embryonated egg,

said method being implemented in the absence of and without adding immune effector cells other than those of the grafted eggs.

The present invention excludes the presence of immune effector cells other than those of the embryonated bird egg into which the tumour cells are grafted.

At no time, the uses and methods according to the present invention can include the presence or addition of immune effector cells other than those of the embryonated egg into which the tumour cells are grafted.

### FIGURE LEGENDS

FIG. 1 represents a model of an embryonated egg with the tumour cell deposition zone and the main tissues present.

FIG. 2 represents an example of the timeline of the investigation from cell grafting to sample collection.

FIG. 3 represents the effect of treatment with atezolizumab (anti-PD-L1 Tecentriq) on tumours initiated from MDA-MB-231 cells.

FIG. 4 represents the effect of treatment with pembrolizumab (anti-PD1 Keytruda) on tumours initiated from (A) MDA-MB-231 cells or (B) SU-DHL-4 cells.

FIG. 5 represents the effect of treatment with RMP1-14 (anti-PD1) on tumours initiated from SU-DHL-4 cells.

FIG. 6 represents the effect of treatment with nivolumab (anti-PD1 Opdivo) on tumours initiated from MDA-MB-231 cells.

FIG. 7 represents the effect of treatment with pembrolizumab (anti-PD1 Keytruda) on metastases in the lower CAM after grafting of MDA-MB-231 cells.

FIG. 8 represents the relative amount (compared to the negative control group) of CD3 (A) and CD4 (B) expression in tumours obtained from SU-DHL-4 cells with or without treatment with atezolizumab (anti-PD-L1 Tecentriq).

FIG. 9 represents the relative amount (compared to the negative control group) of CD3 (A), CD45 (B), CD56 (C) and CD8 (D) expression in tumours obtained from SU-DHL-4 cells with or without treatment with pembrolizumab (anti-PD-1 Keytruda).

FIG. 10 represents the relative amount (compared to the negative control group) of CD3 expression in tumours obtained from MDA-MB-231 cells with or without nivolumab (anti-PD1 Opdivo) treatment.

FIG. 11 represents the different immune cell populations (CD4+T lymphocytes, CD8+T lymphocytes and monocytes) detected by flow cytometry in peripheral blood mononuclear cells from chicken embryos at E16.

FIG. 12 represents the increase in cytotoxic effect of chicken T lymphocytes against human tumour cells H460 after treatment of T lymphocytes with pembrolizumab (anti-PD-1 Keytruda®).

### DETAILED DESCRIPTION OF THE INVENTION

The present invention relates to the use of an embryonated bird egg grafted, in particular at the level of the CAM, with tumour cells to evaluate the anti-cancer activity of one or more immunotherapeutic molecule(s), wherein said model excludes the presence of immune effector cells other than those of the grafted egg. Preferably, the immunotherapeutic molecule is selected from an adoptive cell therapy such as CAR-T, a vaccine, a bi-specific antibody, an immune checkpoint inhibitor such as an anti-PD1, or anti-PDL1, or anti CTLA-4 antibody, and even more preferably from an adoptive cell therapy such as CAR-T, a bi-specific antibody, an immune checkpoint inhibitor such as an anti-PD1, or anti-PDL1, or anti CTLA-4 antibody. Advantageously, the immunotherapeutic molecule is selected from an immune checkpoint inhibitor such as an anti-PD1, or anti-PDL1, or anti CTLA-4 antibody.

The embryonated egg, in particular chicken model, with tumour grafting at the level of the chorioallantoic membrane (CAM) is already widely used for effectiveness and toxicity testing of many types of anti-cancer treatments, such as chemotherapies, peptides or nanoparticles. However, it has never been used to test effectiveness of immunotherapeutic anti-cancer molecules, that is those that resort to activation, or more precisely reactivation, of the cancer patient's own immune system.

The inventors have demonstrated, surprisingly, that this model can be used in the same way to test effectiveness of immunotherapeutic molecules using only the immune system of the grafted egg, although it is very different from that of humans and although many authors may have considered the chicken immune system to be immature and therefore incapable of leading to any immune response. The use of this model according to the invention is therefore implemented in the absence of and without adding immune effector cells other than those of the grafted embryo. This implementation therefore resorts solely to the immune system of the grafted egg. Such an implementation of this model has several advantages over existing models, such as:
the cost (of the egg compared to a mouse and its maintenance in an animal house for several weeks or months);
the presence of a complete immune system, which does not require the presence or addition of effector immune cells other than those of the embryonated grafted egg.

Furthermore, as this is an embryonic model, the immune system is still developing. Nevertheless, the maturation of this immune system is sufficient a few hours after grafting to be activated by therapeutic immune compounds and thus to validate the effectiveness thereof.

Preferably, the embryonated egg according to the invention is an egg of a bird of the order Galliformes or Struthioniformes. In particular, it is particularly preferred that the egg is a gallinaceous egg, especially of chicken, quail, turkey, pheasant, peacock, guinea fowl or other farmyard birds. It may also be an ostrich egg. Advantageously, the embryonated egg according to the present invention is a chicken (Gallus gallus) egg.

Within the scope of the invention, the term “embryonated egg” means a fertilised bird egg in which the embryo can develop under appropriate conditions, in particular in an incubator at a temperature of 37° C. to 38° C. Under these conditions, the incubation time required to hatch the egg is 21 days for the chicken.

The stages of development reported here are defined as a function of the post-fertilisation incubation time of the eggs, in particular the incubation time under the appropriate conditions as defined above.

By “grafting at the level of the CAM” it is intended to designate administration by apposition or injection on the CAM, whether upper CAM or lower CAM.

The embryonated egg model used according to the invention has cells from two different organisms or xenografts: cells from the “host” or “recipient” bird and tumour cells grafted into the egg from a human or animal organism of a different species from that of the “recipient” bird. Particularly preferably, the tumour cells grafted into the embryonated bird egg are human cells. These grafted cells will then develop in the embryo by forming one or more solid tumours and/or by moving in the egg.

According to the invention, grafting the tumour cells is performed in the absence of immune effector cells other than those of the embryonated egg and the use of said egg once grafted excludes the presence and addition of immune effector cells other than those of the grafted egg.

By definition, “grafting at the level of the CAM” takes place once the CAM has formed and at a stage equivalent to at least 8 days of development in the chicken under normal and standard growth conditions. If the bird used is chicken, this stage corresponds to at least 8 days of development. As the number of days of development may vary from species to species, grafting may occur at varying days of development. For example, a stage of development of at least 8 days in chicken corresponds to a stage of development of at least 6.5 days in quail.

It is understood that the grafted embryo used according to the present invention is not intended to hatch and is therefore not intended to create an adult organism. It is only used as an animal model for the duration of the investigation of the effects of immunotherapeutic molecule(s), and not until hatching, which corresponds to 21 days of development in the chicken. In any case, the bird embryo used according to the invention will be sacrificed, according to the ethical rules in force, after the grafted tumour cells have led to the development of one or several tumours in the egg and before hatching.

The grafted tumour cells may be tumour cell lines of different types of cancer, but may also be derived from a tumour sample of a cancer patient, as for example from a biopsy of the patient's tumour or from any other biological sample which contains tumour cells from the patient, provided that the effector immune cells have been removed, that is only the tumour cells have been isolated from the biological sample.

Within the scope of the invention, whether it is upon grafting tumour cell lines or a biological sample from a cancer patient from which the effector immune cells have been removed, no addition of effector immune cells will be made upon using the embryonated egg model or when implementing the methods according to the invention.

According to one embodiment of the invention, the tumour cells obtained from a sample of a patient or animal suffering from cancer are circulating tumour cells (CTC) purified prior to grafting into the embryonated egg. This purification can be achieved by any method known to the skilled person. A large number of different methods have especially been described by Zheyu Shen et al., 2017. They make it possible to achieve so-called “negative” enrichment when the purpose is to capture non-target lymphocytes and elute CTCs, or so-called “positive” enrichment when the purpose is to capture CTCs and elute non-target lymphocytes from the sample. Among these, mention can especially be made of those described in 2013 by Han Wei Hou et al. or again in 2017 by Laget S et al. when these are Circulating Tumour Cells (CTCs), that described in 2013 by Petit Vincent et al. when these are tumour cells isolated from xenografts derived from patients' cells (Patient Derived Xenograft or PDX) and finally those described by DeBord Logan C et al. in 2018.

Preferably, said patient is a human individual. In this case, the sample is a xenograft derived from the tumour of said patient or PDX (patient-derived xenograft).

The tumour cells grafted into the embryonated egg can be in particular from lung cancer, prostate cancer, breast cancer, melanoma, kidney cancer and any other cancer that can benefit from immunotherapy treatment.

Advantageously, the embryonated egg model used according to the invention is a chicken egg into which tumour, preferably human cells, have been grafted at the level of the CAM. Preferably, the use of the grafted chicken egg model excludes the presence of human immune effector cells.

By “immune effector cells” it is meant lymphocytes, in particular T, B and NK lymphocytes, macrophages and dendritic cells.

Within the scope of the present invention, the terms tumour and cancer are used interchangeably and with the same meaning, to define a proliferation of malignant cells. The same applies to the use of the terms antitumour and anticancer.

By “immunotherapeutic molecule” it is intended to designate any compound or product capable of activating an immune response or restoring the action developed by the patient's immune system against his or her tumour. These immunotherapeutic molecules target the functions controlling the immune system that have been blocked by the tumour. Such compounds can be antibodies, especially monoclonal antibodies, adjuvants, chemical molecules etc. In the embryonated egg used according to the invention, the immunotherapeutic molecules are in this case capable of stimulating the immune response of the “host” or “recipient” bird against the cancer that develops from the grafted tumour cells. Among the immunotherapeutic molecules, mention can especially be made of adoptive cell therapies such as CAR-T, vaccines, bi-specific antibodies, immune checkpoint inhibitors such as anti-PD1, or anti-PDL1, or anti CTLA-4 antibodies.

In particular, and when the tumour cells are derived from a cancer patient sample, testing several immunotherapeutic molecules allows selection of the immunotherapeutic molecule that is the most promising for the treatment of the tumour in this patient. Thus, according to one preferred embodiment of the present invention, the embryonated bird egg grafted with tumour cells is used to determine which one has the best anti-cancer activity among the different immunotherapeutic molecules tested.

The embryonated bird egg grafted with tumour cells can also be used according to the present invention to test the anti-cancer effectiveness of combinations of immunotherapeutic molecules in comparison with the effect obtained with each of the molecules tested independently.

Within the scope of the use of this embryonated egg, it is also possible to determine, or even quantify, the toxicity of the immunotherapeutic molecule(s) tested, both on tumours that have developed from the grafted tumour cells and on the embryo as a whole. Accordingly, another object of the present invention relates to the use of an embryonated bird egg grafted with tumour cells to quantify toxicity of one or more immunotherapeutic molecule(s) on the tumour and/or on the embryo as a whole.

According to one preferred embodiment, the uses according to the present invention which are described above are performed with a grafted embryonated bird egg which has been previously incubated to a stage of development corresponding to CAM formation and equivalent to at least 9 or even more preferred 9.5 days of development in chicken.

The present invention also relates to a method for evaluating anti-cancer activity of one or more immunotherapeutic molecule(s), characterised in that it comprises:
grafting tumour cells at the level of the chorioallantoic membrane (CAM) of an embryonated bird egg previously incubated to a stage of development corresponding to CAM formation and equivalent to at least 8 days in the chicken at the time of grafting;
administrating the immunotherapeutic molecule(s) in the embryonated egg at least 12 hours after grafting,
investigating the effect of the immunotherapeutic molecule(s) thus administered on the tumourigenesis of the tumours that have developed in the grafted embryo, and that it is implemented in the absence of and without adding effector immune cells other than those of the grafted egg.

The person skilled in the art will be able to determine the time to graft the tumour cells according to the species of bird used, that is the minimum number of days of incubation or development of the embryonated egg to achieve CAM formation, and at a stage of development equivalent to at least 8 days of development in chickens. For example, in chickens, grafting can take place from 8 days of development, and in quails from 6.5 days of development.

According to one preferred embodiment, the embryonated egg was, prior to grafting, incubated until a stage of development corresponding to CAM formation, and equivalent to at least 9 or even more preferably 9.5 days of development in the chicken.

The incubations are carried out under appropriate conditions, that is conditions which allow the normal development of the embryonated egg, especially at a temperature between 37° C. and 39° C., and preferably 38° C. or even 38.5° C.

Grafting the tumour cells can be performed at any place of the upper or lower CAM, preferably at the level of the upper CAM. Any method well known to the skilled person may be used for this grafting, and in particular, it is possible to use the grafting technique referenced by Crespo P. & Casar B., 2016.

According to one particular embodiment, the amount of tumour cells grafted ranges from about 10 cells to about 5·10⁶ cells.

According to one preferred embodiment, the tumour cells used were frozen prior to grafting into the embryonated egg, either for cell lines or for tumour cells isolated from a cancer patient or animal sample.

In particular, when the grafted tumour cells are derived from a sample of a patient or animal having cancer, testing several immunotherapeutic molecules makes it possible to select the one that is the most promising for treating the tumour in this patient or animal. Thus, according to one preferred embodiment of the present invention, the method for evaluating the anti-cancer activity of one or more immunotherapeutic molecule(s) allows the determination of the immunotherapeutic molecule that exhibits the best anti-cancer activity among the different molecules tested.

The method for evaluating the anti-cancer activity of one or more immunotherapeutic molecule(s) according to the present invention also makes it possible to test the anti-cancer effectiveness of combinations of immunotherapeutic molecule(s) in relation to the effect obtained with each of the immunotherapeutic molecule(s) tested independently.

The step of administering the immunotherapeutic molecule(s) into the embryonated egg can be performed in different ways by techniques well known to the skilled person. The administration can especially be carried out by apposition or injection at the level of the CAM, by intra-tumoural injection, by injection into the embryonic or extra-embryonic structures of the egg.

The administration of the immunotherapeutic molecule(s) is performed at least 12 hours after grafting of the tumour cells, preferably at least 24 hours or even more preferably at least 48 hours after the grafting, that is 1 to 2 days after grafting. The immunotherapeutic molecule(s) can be administered according to different patterns in terms of duration, but also in terms of number of administrations, such as every second day, or every day, or twice a day, or a single injection, until the last day of incubation of the egg. These choices will be determined by the immunotherapeutic molecule administered.

According to one preferred embodiment, the method for evaluating the anti-cancer activity of one or more immunotherapeutic molecule(s) according to the invention, further comprises incubating the embryonated egg once grafted for at least 1 hour, after administrating the immunotherapeutic molecule(s) in the grafted embryonated egg, before investigating the effect on tumourigenesis. Advantageously, the incubation is carried out for at least 4 days and at most 12 days, to correspond to a stage of development of the embryo of at most 21 days, advantageously 18 days of development.

According to one particular embodiment, the method for evaluating the anti-cancer activity of one or more immunotherapeutic molecule(s) according to the invention further comprises collecting the tumours which develop from the grafted tumour cells at the end of the incubation of said embryonated egg after administrating the immunotherapeutic molecule(s) which has (have) been administered, and especially by microdissection.

The investigation of the effect of the immunotherapeutic molecule(s) thus administered on tumourigenesis may take several complementary approaches, in particular after collecting the tumours which have developed in the grafted embryo. It may especially include analysing parameters such as tumour growth, metastatic invasion, angiogenesis, neo-angiogenesis, inflammation and/or tumour immune infiltration, and tumour toxicity.

Tumours can thus be subjected to analyses to measure and/or analyse these different parameters, such as tumour weight and/or volume to investigate tumour growth, expression of different specific markers to investigate metastatic invasion such as Alu sequence amplification by quantitative PCR for human metastasis, number of vessels in the tumour for angiogenesis and neo-angiogenesis, quantification of interleukins for inflammation and/or quantification, especially by rtQPCR, of markers such as CD3, CD8, CD4, CD45 and CD56 to assess tumour immune infiltration, weight, and histological analyses to evaluate the toxicity on the tumour.

The investigation of metastatic invasion can be performed on the lower CAM, which is easily accessible, but it can also be performed in any target organ within the embryo, especially depending on the type of cancer and known data on the associated metastasis phenomenon.

Inflammation and/or tumour immune infiltration can especially be investigated by analysing expression of different markers, such as CD3 (membrane marker for T lymphocytes), CD4 (membrane marker for regulatory T lymphocytes, monocytes and macrophages), CD8 (marker for cytotoxic T lymphocytes), CD45 (membrane marker for leukocytes), CD56 (marker for NK cells), etc. Pairs of oligonucleotides specific to these markers could be developed, in order to avoid inter-species crossing.

By extension, it is also possible to monitor inflammation and infiltration of immune system cells in metastatic sites.

The combined analysis of all these factors, which are well known to the skilled person, makes it possible to determine anti-cancer effectiveness of the immunotherapeutic molecule(s) administered in the embryo. These parameters are especially an integral part of the decision tree used by clinicians to decide on the therapeutic management to be adopted in cancer patients.

Within the scope of all the methods of the invention, which comprise investigating the effect of the immunotherapeutic molecule(s) on tumourigenesis, the anti-cancer activity is preferably evaluated by comparing the tumourigenesis of tumours collected after administrating the immunotherapeutic molecule(s) in the embryonated egg once grafted with that of the tumours collected from another embryonated egg of the same bird previously grafted according to the same method with the same tumour cells but in which no immunotherapeutic molecule has been administered. Similarly, when the effect of several immunotherapeutic molecules is investigated the anti-cancer activity will preferentially be evaluated by comparing the tumourigenesis of tumours collected after administrating the combination of immunotherapeutic molecules in the embryonated egg once grafted with that of the tumours collected from one or more other embryonated eggs of the same bird previously grafted according to the same method with the same tumour cells but in which each of the immunotherapeutic molecules has been administered individually.

Advantageously, it is the anti-cancer activity of one or more immune checkpoint inhibitors that is evaluated within the scope of the method for evaluating anti-cancer activity according to the invention, and preferably the anti-cancer activity of anti-PD1 antibodies or anti-PDL1 antibodies.

The present invention is also concerned with a method for screening immunotherapeutic molecules for cancer treatment in vivo. Thus, according to another aspect, the invention relates to a method for screening immunotherapeutic molecules with anti-cancer activity, comprising the following steps of:
grafting tumour cells at the level of the chorioallantoic membrane (CAM) of an embryonated bird egg previously incubated to a stage of development corresponding to CAM formation and equivalent to at least 8 days of development in the chicken,
administrating the candidate immunotherapeutic molecule(s) into the embryonated egg at least 12 hours after grafting,
investigating the effect of the immunotherapeutic molecule(s) thus administered on the tumourigenesis of the tumours which have developed in the grafted embryonated egg,
   said method being implemented in the absence of and without adding effector immune cells other than those of the grafted egg.

By “candidate immunotherapeutic molecules” it is meant a chemical or biological immunotherapeutic molecule as defined above that is likely to have antitumour/anti-cancer activity, and in particular potentially effective in treating the type of cancer that has developed from the tumour cells grafted into the embryonated egg.

The screening method according to the present invention allows for determining whether or not a candidate therapeutic agent has anti-cancer activity, and whether it has anti-metastasis activity.

According to another aspect, the invention is also concerned with a method for monitoring a patient or animal with cancer, comprising:
preparing a first embryonated bird egg as described above with tumour cells from said patient or animal at a time T1, and investigating the tumourigenesis of the tumours that develop in this first embryonated egg,
preparing a second embryonated bird egg as described above with tumour cells from the same patient or animal at a time T2, and investigating the tumourigenesis of the tumours that develop in this second embryonated egg,
comparing the tumourigenesis of the tumours that have developed in the first and in the second embryonated egg,

said method being implemented in the absence of and without adding effector immune cells other than those of the grafted eggs.

All the above mentioned preferences and details for the method for evaluating the anti-cancer activity of immunotherapeutic molecules apply mutatis mutandis to the monitoring and screening methods according to the present invention.

### Examples

The invention is illustrated hereafter by the use of the hen egg embryonated with a tumour of human origin developed on the chorioallantoic membrane (CAM) to validate the effectiveness of different immunotherapeutic molecules in oncology, and in particular of antibodies directed against two membrane proteins having a major role in the interaction of the immune system with the tumour: PD-1 and PDL-1.

PD-1 (or PDC1 for Programmed Cell Death 1) is a membrane protein expressed on the surface of activated T lymphocytes. Its binding to its ligand, PDL-1 (Programmed Cell Death-Ligand 1), present on the surface of tumour cells leads to an inactivation of T lymphocytes with respect to tumour cells (inhibition of proliferation and cytokine secretion).

Immune checkpoint inhibitors are developed with the aim of removing obstacles that block lymphocytes and prevent them from attacking tumours. Thus, anti-PD1 or anti-PD-L1 antibodies should reactivate the immune system's attack on the tumour.

### Materials & Methods

### Embryos, Incubation Conditions and Grafting

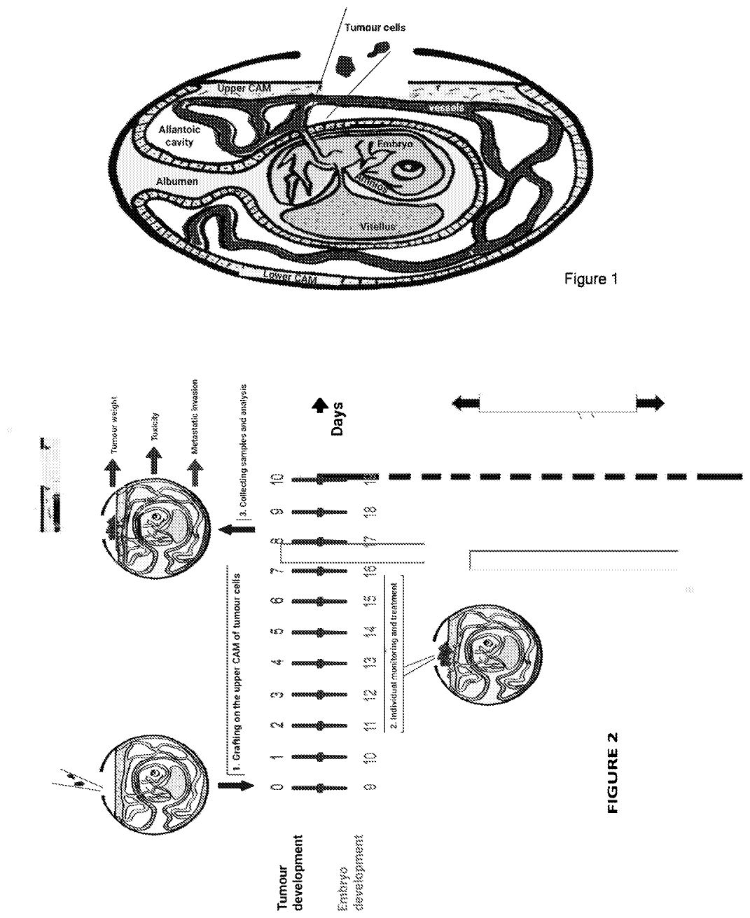

Opening the embryonated egg and grafting tumour cells by apposition on the chorioallantoic membrane (CAM) is already known and widely documented for many years (Crespo P. & Casar B., 2016). A schematic of an embryonated egg with the grafting site of tumour cells on the upper CAM is represented in FIG. 1.

Only the specific conditions used for the validation studies are described here:
fertilised hen eggs were incubated in the supine position for 9.5 days at 37° C. and 40% humidity.
At E9.5 of development, the eggs were opened while preserving the integrity of the CAM. The cells were grafted onto the CAM after opening and then the eggs were re-incubated for 24 hours at 37° C. and 40% humidity.
For each group (control and treated), the results are given on at least 9 eggs (surviving eggs at E18).

### Cell Lines

Standard lymphoma (SU-DHL-4), breast adenocarcinoma (MDA-MB-231), or glioblastoma (U87) cell lines were grafted in the quantities and conditions described in TABLE 1.

TABLE 1

| | | Grafting conditions | |
|---|---|---|---|
| | ATCC | | Amount of |
| Cell line | Reference | Grafting medium | cells |
| MDA-MB-231 | HTB-26 | DMEM + 50% commercial | 1.10⁶ |
| | | extracellular | cells/egg |
| | | matrix(Matrigel; Corning. | |
| | | Ref. 354230) | |
| SU-DHL-4 | CRL-2957 | RPMI-1640 + 50% | 1.10⁶ |
| | | commercial extracellular | cells/egg |
| | | matrix(Matrigel; Corning. | |
| | | Ref. 354230 | |
| U87-MG | HTB-1 | EMEM + 50% commercial | 1.10⁶ |
| | | extracellular | cells/egg |
| | | matrix(Matrigel; Corning. | |
| | | Ref. 354230 | |

### Immunotherapeutic Molecules

After grafting, in ovo tumours were treated with anti-human PD1 or anti-PDL1 antibody on four occasions (E10.5; E12.5; E14.5; E16.5) with 100 μl of antibody (anti-PD1 or anti-PDL1) at different concentrations (detailed in the figures below).

The anti-PD1 and anti-PDL-1 antibodies tested are set out in TABLE 2.

TABLE 2

| Trade name | Antibody | Target | Species |
|---|---|---|---|
| Tecentriq (Roche) | atezolizumab | PDL-1 | human |
| Keytruda (Merck) | pembrolizumab | PD-1 | human |
| Opdivo (Bristol Myers | nivolumab | PD-1 | human |
| Squibb) | | | |
| N/A (BioXcell) | Clone RMP1-14 | PD-1 | mice |

Atezolizumab (anti-PD-L1 Tecentriq) and pembrolizumab (anti-PD-1 Keytruda) are two monoclonal antibodies already prescribed in humans.

Atezolizumab (anti-PD-L1 Tecentriq) is the first anti-PDL-1 to be approved in humans (FDA). It is used against metastatic non-small cell lung cancer. It is also used against urothelial cancer. Pembrolizumab (anti-PD-1 Keytruda) is an anti-PD-1 prescribed against many cancers (melanoma, lung, Hodgkin's lymphoma, prostate, bladder, breast, . . . ), a commercial competitor of nivolumab (Opdivo), which is also an anti-PD-1 but binds to another site on the PD-1 membrane protein (Fessas, P.; Semin Oncol. 2017).

### Collection of Tumours and Analyses

At E18, the eggs were opened, the tumours collected, fixed in 4% Paraformaldehyde in phosphate buffer saline (PBS) cleaned to remove pieces of CAM around the tumour, and then weighed on a precision balance. For metastasis analysis, a piece of the lower CAM (opposite the graft site) can be collected and frozen. This will be used for total genomic DNA extraction. Detection of human cells in these samples is performed by qPCR using specific primers for human Alu sequences (multi-copy sequences well conserved in humans) (Zijlstra, A. et al., 2012).

FIG. 2 represents the timeline of the investigation from cell grafting to sample collection.

The results were obtained in different cancer models, demonstrating the potential of this type of treatment in many models.

### Results

### 1. Effectiveness of Four Immunotherapies on Tumour Growth

### Effectiveness of Atezolizumab (Anti-PDL-1 Tecentriq) on MDA-MB-231 Cells

After cell grafting, the tumours were treated 4 times (E10.5; E12.5; E14.5; E16.5) with 100 μl of atezolizumab (anti-PDL-1 Tecentriq) at a dose of 4 μg/kg per egg.

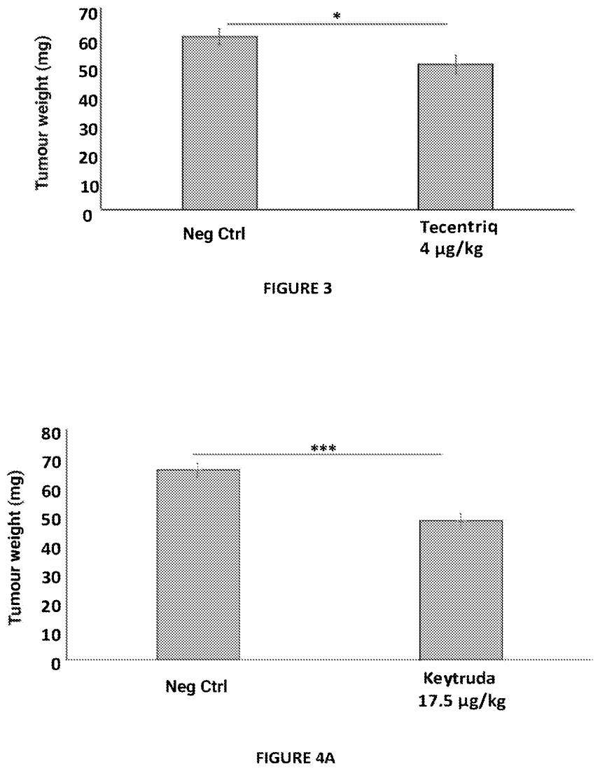

The results of tumour weight analyses initiated from MDA-MB-231 cells, after administrating atezolizumab are pooled in Table 3 (SD: standard deviation; SEM: standard error of the mean) and appended FIG. 3.

TABLE 3

| | | Tumour | | | | P value |
|---|---|---|---|---|---|---|
| | | weight | | | % tumour | vs. Neg |
| | n | (mg) | SD | SEM | regression | Ctrl |
| Neg Ctrl | 17 | 59.98 | 11.602 | 2.814 | N/A | N/A |
| Tecentriq | 13 | 50.30 | 11.523 | 3.196 | 16.13 | 0.03107 |
| (4 μg/kg) | | | | | | |

### Effectiveness of Pembrolizumab (Anti-PD-1 Keytruda) on MDA-MB-231 Cells and SU-DHL-4 Cells

After cell grafting, the tumours were treated 4 times (E10.5; E12.5; E14.5; E16.5) with 100 μl of pembrolizumab (anti-PD-1 Keytruda).

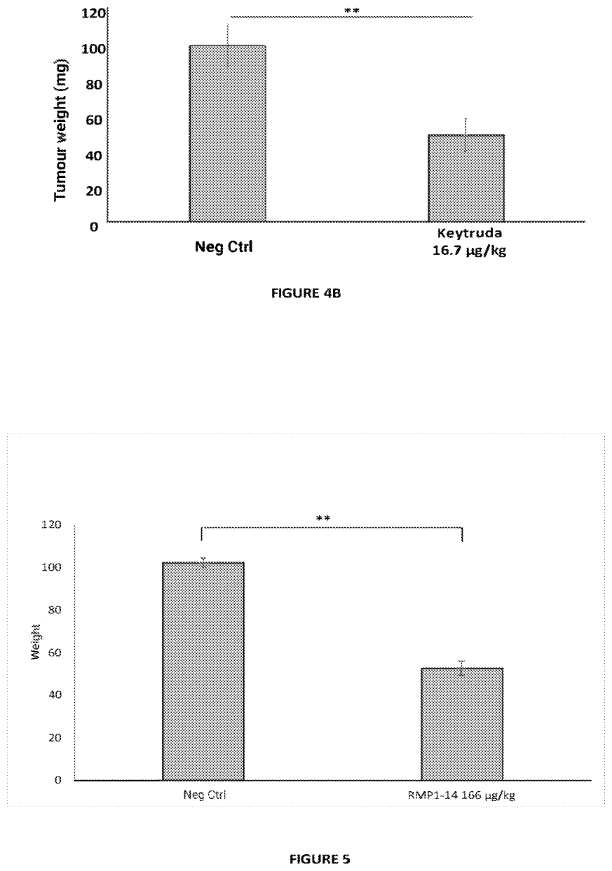

The results of tumour weight analyses initiated from MDA-MB-231 cells, or SU-DHL-4 cells, after administrating pembrolizumab are pooled in TABLES 4 and 5 (SD: standard deviation; SEM: standard error of the mean) as well as in appended FIG. 4.

TABLE 4

| | | Tumour | | | | P value |
|---|---|---|---|---|---|---|
| MDA-MB-231 | | weight | | | % tumour | vs. Neg |
| Cells | n | (mg) | SD | SEM | regression | Ctrl |
| Neg Ctrl | 21 | 66.08 | 11.289 | 2.463 | N/A | / |
| Keytruda | 24 | 48.31 | 12.312 | 2.513 | 26.89 | 0.00001 |
| (17.5 μg/kg) | | | | | | |

TABLE 5

| | | Tumour | | | | P value |
|---|---|---|---|---|---|---|
| SU-DHL-4 | | weight | | | % tumour | vs. Neg |
| Cells | n | (mg) | SD | SEM | regression | Ctrl |
| Neg Ctrl | 13 | 98.92 | 42.037 | 11.659 | N/A | / |
| Keytruda | 9 | 48.77 | 27.238 | 9.079 | 50.70 | 0.00516 |
| (16.7 μg/kg) | | | | | | |

### Effectiveness of Murine Anti-PD-1 RMP1-14 on SU-DHL-4 Cells

After cell grafting, the tumours were treated 4 times (E10.5; E12.5; E14.5; E16.5) with 100 μl of RMP1-14 (murine anti-PDL-1) at a concentration of 166 μg/kg per egg.

The results of tumour weight analyses initiated from SU-DHL-4 cells after administrating RMP1-14 are shown in TABLE 6 (SD: standard deviation; SEM: standard error of the mean) and in appended FIG. 5.

TABLE 6

| | | Tumour | | | | P value |
|---|---|---|---|---|---|---|
| | | weight | | | % tumour | vs. Neg |
| | n | (mg) | SD | SEM | regression | Ctrl |
| Neg Ctrl | 14 | 102.322 | 43.037 | 12.660 | N/A | / |
| RMP1-14 | 12 | 52.646 | 29.659 | 9.125 | 51.451 | 0.00545649 |
| (Anti PD1) | | | | | | |
| (166 μg/kg) | | | | | | |

### Effectiveness of Nivolumab (Anti-PD-1 Opdivo) on MDA-MB-231 Cells

After cell grafting, the tumours were treated 4 times (E10.5; E12.5; E14.5; E16.5) with 100 μl of nivolumab (anti-PD-1 Opdivo).

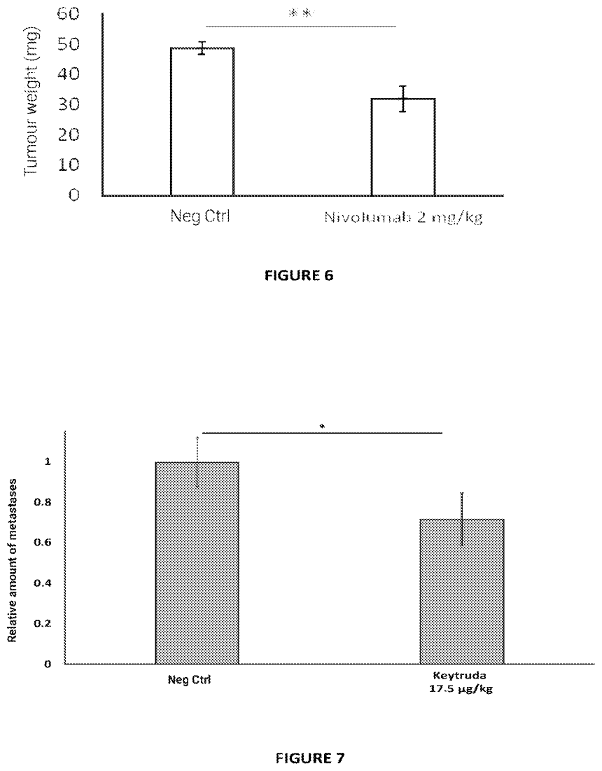

The results of tumour weight analyses initiated from MDA-MB-231 cells after administrating Nivolumab are pooled in TABLE 7 (SD: standard deviation; SEM: standard error of the mean), as well as in appended FIG. 6.

TABLE 7

| | | Tumour | | | | P value |
|---|---|---|---|---|---|---|
| | | weight | | | % tumour | vs. Neg |
| | n | (mg) | SD | SEM | regression | Ctrl |
| Neg Ctrl | 19 | 48.69 | 9.11 | 2.09 | N/A | / |
| Nivolumab | 9 | 31.76 | 12.37 | 4.12 | 34.76 | 0.00036874 |
| (2 mg/kg) | | | | | | |

### 2. Monitoring of Metastatic Invasion

In addition to tumour weight, it is possible to monitor metastatic invasion within the embryonated egg after administrating immunotherapy.

This analysis is conventionally performed on extracted genomic DNA (MagJET Genomic DNA kit; ThermoScientific; Ref.K2721) from any tissue of the embryo or lower CAM by qPCR (Bio-Rad; SsoAdvanced Univ SYBR Green Supermix Ref. 1725274) with oligonucleotides specific to human Alu sequences (multicopy sequences in the human genome) (Zijlstra, A. et al., 2012).

After grafting MDA-MB-231 cells and then administrating pembrolizumab (anti PD-1 Keytruda) as described above, the analysis was herein performed on genomic DNA extracted from a piece of the lower CAM, a readily available tissue.

The results are pooled in appended FIG. 7.

### 3. Analysis of Tumour Infiltration by Immune System Cells

Further to the observed effectiveness on tumour weight, it is possible to analyse the action of immunotherapies by measuring infiltration of immune cells from the embryo into the tumour tissue in the presence or absence of immunotherapies.

The expression of the avian form of the CD3 (membrane marker for T lymphocytes), CD4 (membrane marker for regulatory T lymphocytes, monocytes and macrophages), CD45 (membrane marker for leukocytes), CD8 (cytotoxic T lymphocyte marker) and CD56 (NK cell marker) markers were analysed by qPCR in tumours initiated from SU-DHL-4 cells, after administrating the anti PD-1 pembrolizumab or nivolumab, or the anti-PD-L1 atezolizumab or not, as described above.

Tissues were collected, total RNA extracted (MagJET RNA kit; ThermoScientific; Ref. K2731). From the total RNAs, cDNAs were synthesised (iScript Explore RT and PreAmp Kit; Bio-Rad; Ref. 12004856) with specific pre-amplification for each biomarker sought (PrimePCR, Pre-Amp Assay, Probe Chicken; Bio-Rad; Ref. 10041596). A quantitative PCR is then performed with the oligonucleotides specific to each of the biomarkers (PrimePCR Assay FAM, Chicken; Bio-Rad; Ref. 12001961).

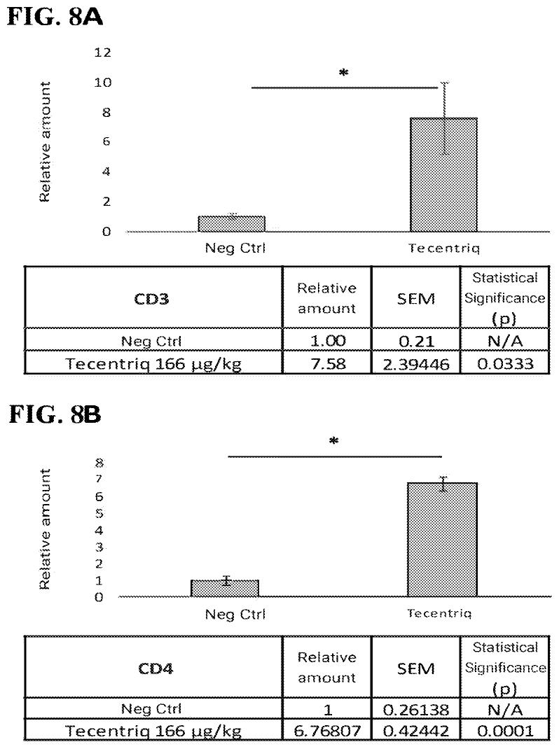

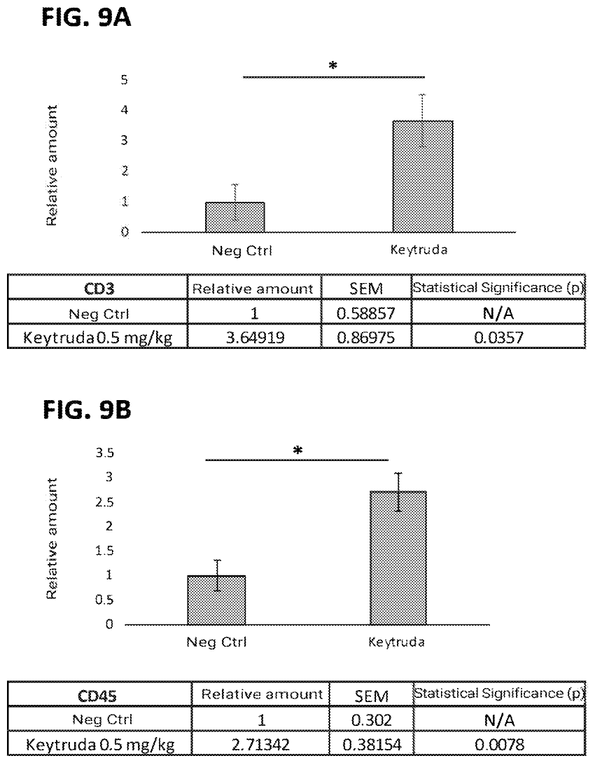

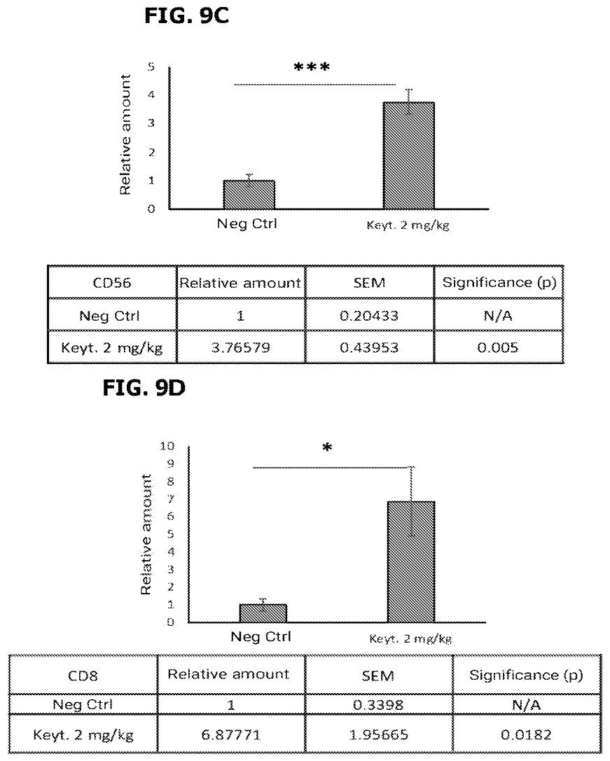

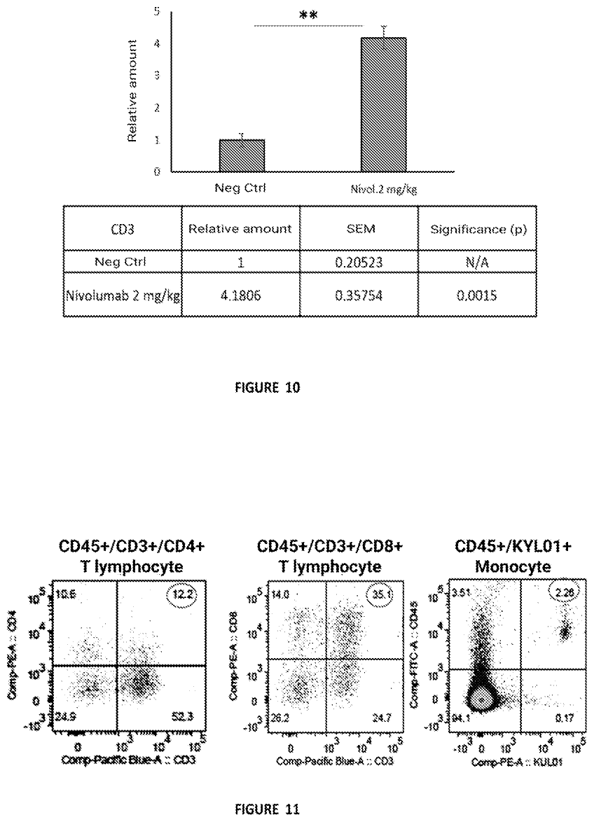

The results are pooled in FIGS. 8 (anti-PDL-1 atezolizumab), 9 (anti-PD-1 pembrolizumab) and 10 (anti-PD-1 Nivolumab) and show the infiltration of immune cells of chicken carrying CD3, CD4, CD45, CD8 and CD56 with or without immunotherapy treatment with either atezolizumab (anti-PDL-1), pembrolizumab (anti PD-1) or nivolumab (anti-PD-1).

4. Characterisation of Chicken Embryo Immune Cells and their Response to Immunotherapy
Characterisation of Immune Cells from Peripheral Blood by Flow Cytometry

Chicken peripheral blood was collected at E16. After purification by Ficoll-Paque® density gradient centrifugation (Sigma-GE17-1440-02), peripheral blood mononuclear cells (PBMC) were labelled with anti-chicken-CD45-FITC (ThermoFisher-MA5-28679), anti-chicken-CD3-Pacific Blue® (CliniSciences 8200-26), anti-chicken-CD8 Alpha-PE (ThermoFisher-MA5-28726), anti-chicken-CD4-PE (ThermoFisher-MA5-28686), anti-chicken KUL01-PE (ThermoFisher-MA5-28828) which identifies chicken monocytes and macrophages. The different immune cell populations were detected by flow cytometry (BD FACSCanto™ II).

The results are pooled in FIG. 11 and show that the different immune cell populations (CD8+T cells, CD4+T cells and monocytes) were detected in the chicken embryo peripheral blood, revealing that the chicken embryo immune system is functional.

### Validation of the Cytotoxicity of Chicken Lymphocytes Against Human H460 Tumour Cells in the Presence of Pembrolizumab (Keytruda) In Vitro

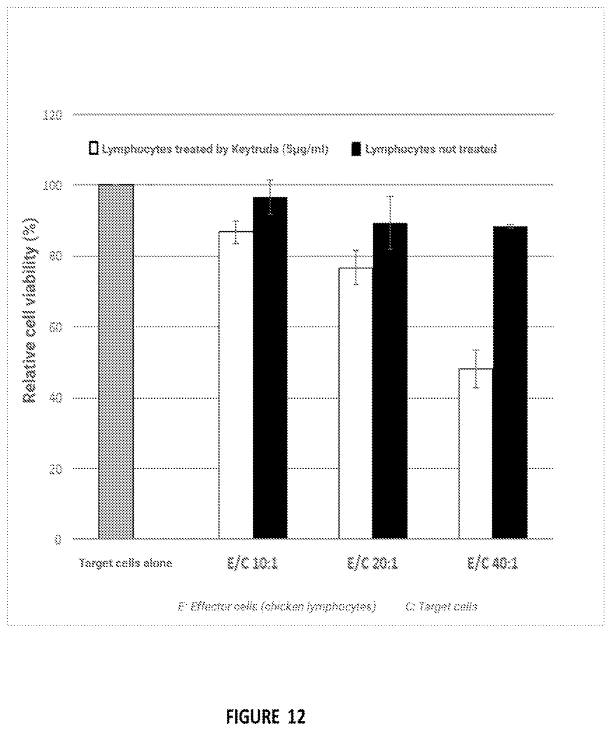

Peripheral blood mononuclear cells (PBMC) from blood collected at E16 as described above were activated by phytohemagglutinin (PHA, Sigma-11249738001, 5 μg/ml) for 72 hours. Then, pembrolizumab (Keytruda®, 5 μg/ml) was added to T lymphocytes maintained in culture for 12 hours to block the PD-1 molecule in order to prevent it from interacting with PD-L1 expressed by the tumour cells. The pembrolizumab-treated and untreated T lymphocytes were then co-cultured with human H460 (lung) tumour cells at different ratios of T lymphocytes (effector cells=E) to tumour cells (target lymphocytes=C): E/C=10:1; E/C=20:1 and E/C=40:1 (FIG. 12). The blockade of PD-1 on chicken T lymphocytes by pembrolizumab was verified by measuring the cytotoxicity of T lymphocytes towards tumour cells as revealed by the in vitro MTT cytotoxicity assay (Sigma-CGD1-1KT).

The results are pooled in FIG. 12 and demonstrate the increase in cytotoxic effect of chicken T lymphocytes against human H460 tumour cells after treatment of T lymphocytes with pembrolizumab.

A larger tumour cell viability was detected when tumour cells were incubated with pembrolizumab-treated T lymphocytes compared to T lymphocytes incubated with tumour cells not treated with pembrolizumab. This difference represents an increase in T lymphocyte cytotoxicity, indicating effective blockade of PD-1 on chicken T lymphocytes by pembrolizumab.

### REFERENCES

Crespo P. & Casar B.; Bio-protocol, Vol. 6, Iss 20, Oct. 20, 2016; Chick Embryo Chorioallantoic Membrane as an in vivo Model to Study Metastasis
Han Wei Hou et al. Scientific Reports 3, Article number: 1259 (2013)
Laget S et al. PloS one, 2017 Jan. 6; 12(1):e0169427.
Petit Vincent et al. Lab Invest. 2013 May; 93(5):611-21.
DeBord Logan C et al. Am J Cancer Res. 2018 Aug. 1; 8(8):1642-1660.
Zijlstra, A. et al. A Quantitative Analysis of Rate-limiting Steps in the Metastatic Cascade Using Human-specific Real-Time Polymerase Chain Reaction. Cancer Res. 62, 7083-7092 (2012)
Fessas, P.; Semin Oncol. 2017 Fessas, P. A molecular and preclinical comparison of the PD-1-targeted T lymphocyte checkpoint inhibitors nivolumab and pembrolizumab; Semin Oncol. 2017 April; 44(2): 136-140. PMID: 28923212).
Zheyu Shen et al, Chem Soc Rev, 2017 Apr. 18; 46(8): 2038-2056. Current Detection Technologies of Circulating Tumour Cells

1. Use of an embryonated bird egg model grafted, in particular at the level of the chorioallantoic membrane (CAM), with tumour cells to evaluate the anti-cancer activity of one or more immunotherapeutic molecule(s) selected from an adoptive cell therapy such as CAR-T, a vaccine, a bi-specific antibody, an immune checkpoint inhibitor such as an anti-PD1, or anti-PDL1, or anti CTLA-4 antibody, wherein said model excludes the presence of immune effector cells other than those of the grafted egg.

2. The use according to claim 1, to select the immunotherapeutic molecule with the most promising anti-cancer activity for the treatment of the tumour developed from the tumour cells grafted into the embryonated egg from the different immunotherapeutic molecules tested.

3. Use of a bird embryonated egg model grafted, in particular at the level of the chorioallantoic membrane (CAM), with tumour cells to determine, or even quantify, toxicity of one or more immunotherapeutic molecule(s) on tumours that have developed from the grafted tumour cells and/or on the embryo as a whole, wherein said model excludes the presence of any immune effector cells other than those of the grafted egg.

4. A method for evaluating anti-cancer activity of one or more immunotherapeutic molecule(s), characterised in that it comprises:
grafting tumour cells at the level of the chorioallantoic membrane (CAM) of an embryonated bird egg previously incubated up to a stage of development corresponding to CAM formation and equivalent to at least 8 days of development in the chicken embryo,
administrating the immunotherapeutic molecule(s) into the embryonated egg at least 12 hours after grafting,
investigating the effect of the immunotherapeutic molecule(s) thus administered on the tumourigenesis of the tumours which have developed in the grafted embryo, and that it is implemented in the absence of and without adding effector immune cells other than those of the grafted egg.

5. The method for evaluating anti-cancer activity of one or more immunotherapeutic molecule(s) according to claim 4, characterised in that it further comprises incubating the embryonated egg once grafted for at least 1 hour, after administrating the immunotherapeutic molecule(s) into the grafted embryonated egg before investigating the effect on tumourigenesis.

6. The method for evaluating anti-cancer activity of one or more immunotherapeutic molecule(s) according to claim 5, characterised in that it further comprises collecting the tumours which develop from the grafted tumour cells at the end of incubation of said embryonated egg after administrating the immunotherapeutic molecule(s).

7. The method for evaluating anti-cancer activity of one or more immunotherapeutic molecule(s) according to any one of claims 4 to 6, characterised in that investigating tumourigenesis comprises measuring various parameters such as tumour growth, metastatic invasion, angiogenesis, neo-angiogenesis, inflammation and/or tumour immune infiltration, and toxicity on the tumour.

8. The method for evaluating anti-cancer activity of one or more immunotherapeutic molecule(s) according to any one of claims 4 to 7, characterised in that the anti-cancer activity is evaluated by comparing the tumourigenesis of the tumours collected after administrating the immunotherapeutic molecule(s) in the embryonated egg once grafted with that of the tumours collected in an embryonated egg of the same bird previously grafted according to the same method with the same tumour cells in which no immunotherapeutic molecule has been administered.

9. A method for screening for immunotherapeutic molecules with anti-cancer activity, comprising the following steps of:
grafting tumour cells at the level of the chorioallantoic membrane (CAM) of an embryonated bird egg previously incubated to a stage of development corresponding to CAM formation and equivalent to at least 8 days of development in the chicken,
administrating the candidate immunotherapeutic molecule(s) into the embryonated egg at least 12 hours after grafting,
investigating the effect of the immunotherapeutic molecule(s) thus administered on the tumourigenesis of the tumours which have developed in the grafted embryonated egg,
said method being implemented in the absence of and without adding effector immune cells other than those of the grafted egg.

10. The screening method according to claim 9, characterised in that it further comprises incubating the embryonated egg once grafted for at least 1 hour, after administrating the immunotherapeutic molecule(s) into the grafted embryonated egg before investigating the effect on tumourigenesis.

11. The screening method according to claim 10, characterised in that it further comprises collecting the tumours that develop from the grafted tumour cells at the end of incubation of said embryonated egg after administrating the immunotherapeutic molecule(s).

12. The screening method according to any one of claims 9 to 11, characterised in that the anti-cancer activity of the candidate immunotherapeutic molecule(s) is evaluated by comparing the tumourigenesis of the tumours collected after administrating the immunotherapeutic molecule(s) in the embryonated egg once grafted with that of the tumours collected in an embryonated egg of the same bird previously grafted according to the same method with the same tumour cells in which no immunotherapeutic molecule has been administered.

13. The method for evaluating anti-cancer activity of one or more immunotherapeutic molecule(s) according to any one of claims 4 to 9 or the method for screening immunotherapeutic molecules having anti-cancer activity according to any one of claims 9 to 12, characterised in that said tumour cells grafted into the bird embryonated egg are derived from a sample of a patient or animal having cancer.

14. A method for monitoring a patient or animal having cancer, comprising:
preparing a first embryonated bird egg by grafting at the level of the chorioallantoic membrane (CAM) tumour cells derived from said patient or animal at a time T1, previously incubated to a stage of development corresponding to CAM formation and equivalent to at least 8 days of development in the chicken, and investigating the tumourigenesis of the tumours which develop in this first embryonated egg,
preparing a second embryonated bird egg by grafting at the level of the chorioallantoic membrane (CAM) tumour cells from said patient or animal at a time T2, previously incubated until a stage of development corresponding to CAM formation and equivalent to at least 8 days of development in the chicken, and investigating the tumourigenesis of the tumours which develop in this second embryonated egg,
comparing the tumourigenesis of the tumours that have developed in the first and second embryonated egg; said method being implemented in the absence of and without adding effector immune cells other than those of the grafted eggs.
